(19) United States
(12) Patent Application Publication
(10) Pub. No.: US 2013/0045926 A1
(43) Pub. Date: Feb. 21, 2013

(54) COMPOSITION AND METHODS FOR THE PREVENTION AND TREATMENT OF MACULAR DEGENERATION, DIABETIC RETINOPATHY, AND DIABETIC MACULAR EDEMA

(71) Applicants: Dale P. Devore, Chelmsford, MA (US); Bruce H. Dewoolfson, Vienna, VA (US); Vance Thompson, Sioux Falls, SD (US)

(72) Inventors: Dale P. Devore, Chelmsford, MA (US); Bruce H. Dewoolfson, Vienna, VA (US); Vance Thompson, Sioux Falls, SD (US)

(21) Appl. No.: 13/512,972

(22) PCT Filed: Dec. 3, 2010
(86) PCT No.: PCT/US10/58856

§ 371 Date: Aug. 6, 2012

Related U.S. Application Data
(60) Provisional application No. 61/329,410, filed on Apr. 29, 2010.
(60) Provisional application No. 61/266,705, filed on Dec. 4, 2009.

(51) Int. Cl. A61K 38/17; A61P 27/02

(52) U.S. Cl. 514/20.8

(57) ABSTRACT

Methods of stabilizing and organizing collagen fibrils in extracellular matrix of retinal tissues, particularly Bruch's membranes, and stabilizing retinal pigment epithelial layers lining Bruch's membrane are disclosed. The stabilization and organization may be effected by treating retinal tissues with a protein that crosslinks and organizes collagen fibrils, such as decorin. The stabilization and organization methods include treatment of retinal tissues before, during, or after diagnosis of dry macular degeneration, diagnosis of early stages of diabetic retinopathy and diabetic macular edema to prevent, retard, or limit progression of disorganization of Bruch's membrane and disorganization of retinal pigment epithelial cells lining Bruch's membrane.

### I. RELATED APPLICATIONS

This application claims priority to U.S. Provisional Application No. 61/266,705, filed Dec. 4, 2009 and U.S. Provisional Application No. 61/329,410, filed Apr. 29, 2010, the disclosures of each of which are incorporated by reference in their entirety.

### II. FIELD OF THE INVENTION

The present invention relates to chemical compositions suitable for application to retinal tissues of the eye to strengthen and organize, or re-organize, the extracellular matrix structure of retinal tissues resulting in tissue stabilization; and stabilization and protection of the retinal pigment epithelial cell layer lining Bruch's membrane to retard or prevent the development of macular degeneration, diabetic retinopathy and diabetic macular edema. More particularly the present invention relates to chemical compositions suitable for strengthening and organizing the extracellular matrix structure of Bruch's membrane and stabilizing and protecting the retinal pigment epithelial layer lining Bruch's membrane to maintain the integrity of the barrier between the choroids and the retina, thereby preventing the disruption of Bruch's membrane that leads to (1) detachment and death of retinal epithelial cells, (2) development of choroidal neovascularization, and (3) progressive development of age related macular degeneration.

### III. BACKGROUND

Age-related macular degeneration (AMD) is the most common, chronic degenerative macular disorder. It is the number one cause of legal blindness in the United States in persons over 65 years of age and is present in 10% of the population over the age of 52 years and in 33% of those over 75 years of age.

It has been reported that 20 to 25 million people are affected with AMD worldwide and the numbers continue to increase with the rapid growth in the aged population. It is expected that the current numbers will triple in the next 30-40 years.

Two forms of AMD exist: wet type (exudative or vascular) and dry type (non-exudative, atrophic or non-neovascular). The wet type, which is less common than the dry type, occurs when new vessels form to improve the blood supply to oxygen deprived retinal tissue. However, the new vessels are very delicate and break easily, causing bleeding and damage to surrounding tissue. Loss of central vision can occur suddenly and, if untreated, may result in so-called disciform degeneration. Other symptoms include distortion, decreased contrast sensitivity, and decreased color vision. Wet AMD is thought to result from biochemical and structural changes in Bruch's membrane associated with dry AMD.

The dry type is much more common and is characterized by drusen and loss of pigment in the retina. Drusen are small, yellowish deposits that form on or around Bruch's membrane. Loss of vision is more gradual and less severe than in the wet type. However, a small number of patients with dry macular degeneration eventually develop geographic atrophy of the retinal pigment epithelium, a form of dry AMD associated with substantial reductions in best corrected visual acuity.

Unfortunately, there is no proven treatment for dry AMD. The results of several recent trials do not support the use of laser for dry AMD. Doctors usually watch or monitor dry AMD for the first signs that it is progressing to the more dangerous wet-AMD. As stated above, treatment for wet AMD is most successful when treatment occurs in the early stages. It may even be possible to prevent development of AMD if an appropriate treatment can be identified.

### AMD

As noted above, there are two distinct forms of AMD, dry and wet. The dry form is more common than the wet, with about 90% of AMD patients diagnosed with dry AMD. The wet form of the disease usually leads to more serious vision loss.

Wet AMD affects approximately 10% of people with AMD, but accounts for approximately 90% of all severe vision loss from AMD (National Eye Institute).

With wet AMD, new, poorly-formed blood vessels grow beneath the retina (from the choroids) and leak blood and fluid into the retina and subretinal space. This leakage causes retinal cells to die, promotes scarring of the fovea (central macula), and the scarring creates blind spots in central vision.

The dry form is characterized by drusen and loss of pigment in the retina. Drusen are small, yellowish deposits that from on or around Bruch's membrane.

The retina is a complex multilayered structure that can be functionally be considered in two sections; the photosensitive layer of rods and cones and their neural connections that gather light and convert it to electrical nerve impulses transmitted by the optic nerve and the underlying retinal pigment epithelium and underlying basal lamina called Bruch's membrane, which work together to maintain the integrity of the barrier between the choroids and the retina. The choroids, a main vascular tunica, is found between the retina and the sclera and provides the main source of blood supply to the outer half of the retina.

### Aging and AMD

Aging is associated with biological changes throughout the body, including the eye. While aging changes may or may not be associated with AMD, it is particularly important to recognize changes in the junction between retinal pigment epithelium and Bruch's membrane that occur during aging. It is known that aging is associated with alterations in the extracellular matrix (ECM) compositions and structure. Bruch's membrane lamina is composed of four ECM layers, each containing collagens, glycosaminoglycans, and glycoproteins. During aging, changes occur in Bruch's membrane, including overall thickening of the inner collagenous layer and changes in collagen content and collagen spatial organization. Crosslinking appears to increase, accompanied by an increase in noncollagen protein in the submacular Bruch's membrane. In some investigations an increase in glycosaminoglycan size and content has been reported. Overall, changes in the composition and structure of Bruch's membrane lead to a change in permeability and diffusion through this membrane. Overall, abnormal ECM of AMD eyes is characterized by basal laminar deposit, basal linear deposit, and their clinically evident manifestation, soft drusen.

### Diabetic Retinopathy

Diabetic retinopathy is a microangiopathy of the retina and involves capillary leakage and retinal ischemia. Multiple biochemical pathways are involved including production of vascular endothelial cell growth factor (VEGF) that causes neo-vascularization, increased vascular permeability, and collapse of the blood-retinal barrier. Progression of diabetic retinopathy is age-related and associated with structural and biochemical changes in Bruch's membrane (BM).

Diabetic retinopathy (DR), a leading cause of blindness in type 1 and type 2 diabetics, is a complication of diabetes which produces damage to the blood vessels inside the retina. Diabetic retinopathy can have four stages: (1) mild nonproliferative retinopathy, wherein microaneurysms in the retina's blood vessels occur; (2) moderate nonproliferative retinopathy, wherein some blood vessels feeding the retina become blocked; (3) severe nonproliferative retinopathy, wherein many blood vessels to the retina are blocked, depriving several areas of the retina with their blood supply; and (4) proliferative retinopathy, wherein new, abnormal, thin-walled and fragile-walled blood vessels grow to supply blood to the retina, but which new blood vessels may leak blood to produce severe vision loss and blindness. Hemorrhages can occur more than once, often during sleep. Fluid can also leak into the center of the macula at any stage of diabetic retinopathy and cause macular edema and blurred vision. About 40 to 45 percent of Americans diagnosed with diabetes have some stage of diabetic retinopathy, and about half of the people with proliferative retinopathy also have macular edema.

The pathogenesis and blindness associated with ocular diseases such as diabetic retinopathy and age-related macular degeneration is a direct outcome of unwanted angiogenesis. In ocular tissue, angiogenesis is the most common cause of blindness. Proliferative diabetic retinopathy is characterized by retinal blood vessel incursion of the vitreous. Age-related macular degeneration is a disease of the macula and is distinguished from the dry form by choroidal blood vessel in-growth penetrating Bruch's membrane.

### Diabetic Macular Edema

Diabetic Macular Edema (DME) is described as a thickening of the retina and/or hard exudates within 1 disc diameter of the center of the retina. DME and Diabetic Retinopathy (DR) are microvascular complications in patients with diabetes that have debilitating impacts on visual acuity, eventually leading to blindness. Patients with DR can develop DME and DME occurs after breakdown of the blood-retinal barrier because of leakage of dilated hyperpermeable capillaries and microaneurysms. Like DR, DME is associated with choroidal neovascularization penetrating damaged or disorganized Bruch's membrane.

### Angiogenesis

Angiogenesis (also referred to herein as neovascularization) is the process whereby new blood vessels are formed. Angiogenesis occurs normally during embryogenesis and development, and occurs in fully developed organisms during wound healing and placental development. In addition, angiogenesis occurs in various pathological conditions, including in ocular diseases such as diabetic retinopathy and macular degeneration due to neovascularization. Recent studies have suggested that alterations in Bruch's membrane accompanied by changes in the extracellular matrix of RPE cells may contribute to the increased production of VEGF in patients with choroidal neovascularization (Kwak, et al.). Furthermore, animal studies (Kwak, et al) confirm that normal RPE cells and intact Bruch's membrane provide a physical or biochemical barrier to vascular invasion from the choroid.

### Bruch's Membrane

Bruch's membrane is a pentalaminar structure composed of the RPE basement membrane, inner collagenous layers, middle elastic layer, and outer collagenous layers. This extracellular matrix meshwork between the Retina Pigment Epithelial (RPE) cells and the choroid is 2-4 μm is thickness and is known to undergo structural changes and chemical reconfiguration during aging. Bruch's membrane is under constant cycles of pressure-induced stress as a result of choroidal flow oscillating with the cardiac rhythm and the mechanical properties of Bruch's membrane are critical to its physiology and ability to function as an effective barrier between adherent RPEs and the vascularized choroid. Studies have shown that the elasticity of Bruch's membrane decreases linearly with aging after the age of 21, with an approximate reduction of 1% per year.

### Bruch's Membrane and Retinal Pigment Epithelial Cells

In normal eyes, retina pigment epithelium (RPE) forms a hexagonal cell monolayer lining Bruch's membrane internally that separates the neural retina from the choroiocapillaris (choroid). RPEs are responsible for maintaining the integrity of the neural retina, choriocapillaris, and Bruch's membrane. The integrity of the RPE layer is maintained as long as there is proper attachment to Bruch's membrane. While the correlation between age-related structural changes in Bruch's membrane and cellular changes in RPEs are unknown, it is known that structural changes in Bruch's membrane may precede cellular changes in RPE by one or two decades and can induce changes in attachment, survival, proliferation, and gene expression profiles of the overlying RPE. At this time it is not specifically known which structural or chemical changes in Bruch's membrane are responsible for age-dependent effects on RPE. It is know that there are numerous changes within Bruch's membrane with age including deposition of abnormal proteins and lipids, topographical reorganization of protein matrix structure, and changes in the ligand binding sites necessary for cell attachment.

Treatments to Modify Bruch's membrane

There have been attempts to alter Bruch's membrane by systemic dialysis (Fell, A J, et.al.) and to repair (Del Priore, L) or reconstruct (Tezel, T H, et.al.) Bruch's membrane to restore normal barrier function and increase RPE attachment. If successful, Bruch's membrane would regain its ability to prevent choroidal neovascularization (CNV).

Over the last several years, strategies for eliminating abnormal blood vessels under the central retina (macula) have been shown to help a significant proportion of patients with the ‘wet’ form of age-related macular degeneration (AMD). Unfortunately, these vessels often recur because the underlying structural defects in Bruch's membrane are not repaired. In addition, no treatment is available for the restoration of the retinal pigment epithelium (RPE) in patients with the ‘dry’ form of AMD (without abnormal blood vessels), again because of underlying defects in Bruch's membrane that prevent RPE cells from adhering to this structure to reform an intact monolayer. In either case, a method is needed for local restoration of the integrity of Bruch's membrane that will prevent the ingress of neovascular anomalies and/or allow the reconstitution of the RPE monolayer, either by host or grafted cells.

Local submacular repair of Bruch's membrane is therefore fundamental to restoration of the RPE monolayer and preservation of the adjacent photoreceptors (rods and cones) that are essential for vision. Up until now, experimental attempts to repair Bruch's membrane have been frustrated by a number of significant challenges. These challenges include the need for a material that does not induce an inflammatory or foreign body response when implanted beneath the retina and/or RPE, the need for a material construction that allows RPE cells to adhere and grow as an undistorted monolayer while also not disturbing the precise organization of the overlying photoreceptor outer segments, the need for the material to be sufficiently thin and porous for maintaining normal structural relationships in the macula and for diffusion of physiologically important molecules between choroid, RPE, and retina, the need for the material to be resilient with sufficient elasticity and not be overly brittle so that it can be surgically delivered intact to the subretinal space. Another desirable quality is biodegradability.

Patent Publication 20090306772 describes the implantation of a temporary structural barrier between the RPE and the underlying choriocapillaris, the scaffold serving as a template to allow these host structures to lie down and maintain a new basement membrane structure effectively similar to native Bruch's membrane. The invention describes methods for in situ repair of Bruch's membrane, the structure underlying the RPE in the eye and constituting the site of early, fundamental damage in both the exudative (wet) and atrophic (dry) forms of AMD. In certain embodiments, the invention employs polymeric scaffolds for the treatment of retinal disease through implantation of these structures in the subretinal space of a subject (e.g., human subject). It is believed that by forming a temporary structural barrier between the RPE and the underlying choriocapillaris, the scaffold serves as a template to allow these host structures to lie down and maintain a new basement membrane structure effectively similar to native Bruch's membrane.

### Composition of ECM in Outer Layers of The Eye

Extracellular Matrix tissue (ECM) is predominant in Bruch's membrane, the multi-layered connective tissue located beneath retinal photoreceptors and retinal pigment epithelium. Bruch's membrane is composed of collagens, glycosaminoglycans, proteoglycans, and glycoproteins. Collagens include Types I, III, IV, V, and VI. Proteoglycans include decorin. Glycosaminoglycans include chondroitin sulfate and dermatan sulfate. Glycoproteins include laminin and fibronectin.

Decorin has been identified in retinal tissues, and in Bruch's membrane, and it is reported to be important in the development of Bruch's membrane (Hirabayashi, et.al., Medical Electron Microscopy, 36: 136-146, 2003) and in differentiation of retinal ganglion cells (Inatani, et.al., Investigative Ophthalmology & Visual Sci., 40: 1783-1791, 1999).

### Small Leucine-Rich Proteoglycans

Corneal collagen fibrils are associated with natural binding macromolecules including fibril-associated collagens (FACIT), such as Types XII, XIV, and XX collagen, and small leucine-rich repeat proteoglycans (SLRP) such as decorin, lumican, keratocan, fibromodulin, and epiphycan. These macromolecules interact with collagen fibrils to control fibril diameter and stabilize ECM organization. They may also be involved in binding fibrils together to limit the ability of fibrils to glide past one another. These bridges allow some flexibility but limit the distance that fibrils can move. It may be possible to add decorin to ECMs to form bridges between collagen fibrils and thus stabilize ECM. The addition of proteoglycans has been shown to prevent abnormal collagen fibril “growth” resulting from prior removal of proteoglycans.

### Decorin

Decorin is a member of a family of small leucine-rich repeat proteoglycans or SLRPS. Decorin is an approximately 100 kDa proteoglycan consisting of a 40 kDa core protein and one chondroitin sulfate or dermatan sulfate glycosaminoglycan chain. Decorin interacts with collagen Type I and II, fibronectin, thrombospondin and TGF_{β}. Commercial, animal derived decorin can be obtained from Sigma Chemical Company and other suppliers of biochemicals. However, the present inventors procure recombinant human decorin protein from Catalent Pharma Solutions (known as Galacorin™). Decorin core protein has recently been shown to interact with four to six collagen molecules (Orgel, et.al., PlosOne, 4:1-10, 2009) and to stabilize inter-fibrillar organization of extracellular matrix structures.

Decorin functions as a tissue stabilizer and organizer. Decorin is a horseshoe shaped proteoglycan that binds to collagen fibrils in human cornea forming a bidentate ligand attached to two neighboring collagen molecules in the fibril or in adjacent fibrils, helping to stabilize fibrils and orient fibrillogenesis (Scott, J E, Biochemistry, 35: 8795, 1996). Decorin appears to be a ubiquitous component of extracellular matrices linking collagen fibrils at specific binding sites (Scott, J E, et.al., Exp. Cell Res., 243: 59-66, 1998). Corneal transparency is dependent on the size and arrangement of collagen fibrils in the corneal stroma. Decorin binding is critical in limiting collagen fibril growth and in controlling the arrangement of collagen fibrils to produce a transparency (Rada, J A, et.al., Exp Eye Res., 56: 635, 1993.). Studies of effects of decorin on in vitro collagen fibrillogenesis show inhibition of collagen fibril growth and increase in collagen fibril diameter (Kuc, IM and Scott, PG, Connective Tissue Res., 36: 287, 1997).

Decorin also has a number of biological/physiological characteristics including; biological ligand for the EGF receptor and possible regulation of cell growth (lozzo, RV, et.al., J. Biol. Chem., 274: 4489, 1999), down-regulation of TGF_{β}(Giri, SN, et.al., Biochem. Pharmacol., 54: 1205, 1997; Westergen-Thorsson, G., et.al., J. Clin. Invest., 92: , 632, 1993), inhibition of cell attachment and possible anti-adhesion effects (Gu, J. and Wada, Y., J. Biochem (Tokyo), 119: 743, 1996), inhibition of cancer cells (Nash, M A, et.al., Cancer Res., 59: , 6192, 1999) and inhibition of angiogenesis (Grant, et.al., Oncogene, 21: 4765-4777, 2002; Sulochana, et.al., J. Biol. Chem. 280:27935-27948, 2005).

### Treatment of AMD

As discussed above, there are no proven treatments for dry AMD. However, there are several approaches being investigated including preventing the loss of photoreceptors and retinal pigment endothelial cells using encapsulated ciliary neurotrophic factor (CNTF)-producing cells, inhibiting the complement cascade using POT-4 (inhibits complement component C-3) and Eculizumab (infused intraveneous to inhibit), and treating blood using a rheopheresis blood treatment that uses blood filters to deplete excesses of large proteins, fats and other substances from the blood, thereby improving blood flow to the macula and potentially improving vision. Other proposed methods for treating dry AMD include consumption of antioxidant and zinc supplements.

Unlike dry AMD, there are several agents approved by the FDA or under investigation to treat wet AMD. These agents primarily act by inhibiting angiogenesis through the blockage of vascular endothelial growth factor (VEGF). These agents include Pegaptanib Sodium (Macugen®), Ranibizumab (Lucentis®), Bevacizumab (Avastin®), and Anecortave acetate (Retaane®).

### Patent References

The effectiveness of decorin in stabilizing the corneal stroma following orthokeratology procedures to redistribute corneal tissue to correct myopia and hyperopia is described in U.S. Pat. No. 6,946,440 and 7,402,562 and in Patent Publication US2009/0105127. In these patents, the inventors discovered that decorin, other small leucine-rich proteoglycans, and fiber associated collagens with interrupted triple helices molecules, could be delivered into the cornea in the front of the eye, to crosslink stromal collagen fibers to stabilize redistributed corneal tissue following orthokeratology. In the present invention, the inventors have discovered that decorin can be applied to tissues in the back of the eye to maintain the extracellular matrix organization, prevent disorganization of the extracellular matrix, or reorganize the extracellular matrix of Bruch's membrane. Decorin applications for maintaining the organization of Bruch's membrane are clearly differentiated from the previous applications for corneal stabilization. For example, administration of decorin to the cornea is intended to increase its biomechanical stability by forming bridges between the well organized, collagenous, lamellar sheets of the stroma. Delivery to Bruch's membrane is intended to maintain and stabilize matrix organization or to reorganize the matrix structure of one or more layers of this diverse structure comprised of at least five unique, collagenous layers.

Delivery of decorin to the back of the eye cannot be accomplished by topical administration to the cornea for several reasons; the molecule is too large to diffuse readily through the tightly organized corneal epithelium and endothelium, and more importantly, decorin will spontaneously bind to specific amino acid sequences on collagen molecules in the corneal stroma. Therefore, in those application involving delivery to Bruch's membrane, decorin must generally be directly administered to the back of the eye or to the outer layers of the back of the eye by minimally invasive subconjunctival injection, traditional intravitreal injection, transcleral delivery, sub-tenon's injection, supra-choroidal delivery, or by other appropriate methods.

WO2005116066 describes decorin peptides useful for inhibiting undesirable angiogenesis (also published in Sulochana ,et.al 2005). Based on the angiogenesis inhibiting properties of the peptides the authors propose that the decorin peptides can be used to treat macular degeneration. Similarly, US 2009/0246133 A1 describes compositions and methods useful for targeting tissue undergoing angiogenesis, including conjugates composed of an angiogenesis targeting moiety combined with a therapeutic moiety that includes decorin, useful for treating diabetic retinopathy and age-related macular degeneration.

While it is known that decorin exhibits anti-angiogenic activity and might be useful to treat diabetic retinopathy, diabetic macular edema and AMD associated with angiogenesis (i.e., wet AMD), the inventors have found that decorin, including recombinant human decorin core protein can also be used to treat or prevent dry AMD. Without being bound to the particular theory, it is believed this treatment or prevention is possible because decorin stabilizes and organizes (or reorganizes) the extracellular matrix (ECM) in Bruch's membrane to prevent development of abnormal ECM. Abnormal ECM eventually leads to basal laminar and basal linear deposits and soft drusen formation and dissociation of the Bruch's membrane. It is only at this later stage that blood vessels can sprout from the choriocapillaris and break through into sub-RPE and sub-retinal spaces leading to the neovascularization observed in wet AMD, diabetic retinopathy, and diabetic macular edema. Thus, treatment of dry AMD does not involve decorin's anti-angiogenic property. The inventors have also shown that decorin when injected into the vitreous of an animal model is not associated with any adverse observations. Accordingly, the present invention describe compositions and methods for stabilizing and organizing retinal tissue collagen-containing extracellular matrix to maintain the structure and organization of Bruch's membrane thereby preventing, retarding, or limiting the progression of disorganization of Bruch's membrane.

### IV. SUMMARY OF THE INVENTION

In accordance with the invention, compositions and methods for treating or preventing dry AMD, preventing development of wet AMD, and preventing or delaying diabetic retinopathy and diabetic macular edema are disclosed. In particular compositions and methods of stabilizing and maintaining organization or of reorganizing and stabilizing ECM in retinal tissue, including Bruch's membrane are disclosed. These methods comprise administering to the eye of a patient a composition comprising a small leucine-rich proteoglycan (SLRP) molecule or the protein component of a SLRP molecule that crosslinks collagen fibrils and stabilizes inter-fibrillar organization, contained in a pharmaceutically acceptable carrier. In one embodiment of the invention, a protein, such as decorin core protein, crosslinks the collagen fibrils by binding to at lease two different fibrils, and up to six collagen molecules, to form a bridge there between. In another embodiment of the invention, a proteoglycan, such as decorin, biglycan, epiphycan, keratocan, mimican, fibromodulin, lumican or any combination thereof, crosslinks collagen fibrils and stabilizes inter-fibrillar and cellular organization.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### V. DESCRIPTION OF THE EMBODIMENTS

The inventors have found that collagen fibrils in tissue ECM in the outer layers of the eye, particularly in the multilayered Bruch's Membrane, can be stabilized and organized, or reorganized, by administering to the eye one or more proteoglycan molecules or proteins that crosslink and organize the collagen fibrillar structure. In addition, the inventors have found that endothelial and epithelial cell layers in ocular tissues can be stabilized by administering to the eye one or more proteoglycan molecules or proteins. In order that the present invention may be more readily understood, certain terms are first defined. Other definitions are set forth throughout the description of the embodiments.

### Definitions

“Age related macular degeneration”—degeneration of the macula, which is the part of the retina responsible for the sharp, central vision needed to read or drive. Because the macula primarily is affected in AMD, central vision loss may occur. Two forms of AMD exist: wet type (exudative or vascular) and dry type (non-exudative, atrophic or non-neovascular).

Diabetic Retinopathy—Diabetic retinopathy is a microangiopathy of the retina and involves capillary leakage and retinal ischemia. Multiple biochemical pathways are involved including production of vascular endothelial cell growth factor (VEGF) that causes neo-vascularization, increased vascular permeability, and collapse of the blood-retinal barrier. Progression of diabetic retinopathy is age-related and associated with structural and biochemical changes in Bruch's membrane (BM).

Diabetic Macular Edema—Diabetic Macular Edema (DME) is described as a thickening of the retina and/or hard exudates within 1 disc diameter of the center of the retina. DME and Diabetic Retinopathy (DR) are microvascular complications in patients with diabetes that have debilitating impacts on visual acuity, eventually leading to blindness. Patients with DR can develop DME and DME occurs after breakdown of the blood-retinal barrier because of leakage of dilated hyperpermeable capillaries and microaneurysms. Like DR, DME is associated with choroidal neovascularization penetrating damaged or disorganized Bruch's membrane.

“Macula”—is a small and highly sensitive part of the retina responsible for detailed central vision.

“Bruch's Membrane”—is the innermost layer of the choroid consisting of

five layers comprised of extracellular matrix components.

“Retina”—is a light sensitive tissue lining the inner surface of the back of the eye. The optics of the eye creates an image of the visual world on the retina, which serves much the same function as the film in a camera.

“Retinal tissue”—is a term used to encompass the retina and its associated tissues, including Bruch's membrane and the choroids.

“Choroids”—is the vascular layer containing connective tissue of the eye lying between the retina and the sclera.

“Sclera”—is the opaque, white part of the eye or the fibrous, protective, outer layer of the eye containing collagen and elastic fiber.

“Angiogenesis”—is a physiological process involving the growth of new blood vessels from pre-existing vessels.

“Neovascularization”—is the formation of functional microvascular networks with red blood cell perfusion.

“Choriocapillaris”—is a layer of capillaries that is immediately adjacent to Bruch's membrane in the choroid.

“Stabilizing” -includes increasing the tissue rigidity or resistance to stress. “Stabilizing” can also mean decreasing the ability of one collagen fibril to move relative to another collagen fibril by virtue of increased intermolecular interactions.

“Extracellular Matrix or ECM”—is the extracellular part of animal tissue that usually provides structural support to the animal cells in addition to performing various other important functions. The extracellular matrix is the defining feature of connective tissue in animals. ECM contains collagens, proteoglycans, glycosaminoglycans, elastin, glycoproteins, and hyaluronic acid.

“Crosslinks”—includes the formation of both direct and indirect bonds between two or more collagen fibrils. Direct bonds include covalent bond formation between an amino acid in one collagen fibril and an amino acid in another fibril. For example, decorin is a horse-shoe shaped proteoglycan that binds to collagen fibrils in human cornea forming a bidentate ligand attached to two or more neighboring collagen molecules in the fibril or in adjacent fibrils, helping to stabilize fibrils and orient fibrillogenesis. Scott, J E, Biochemistry, Vol. 35, pages 8795 (1996).

A “protein that crosslinks collagen fibrils” includes proteins that form direct or indirect crosslinks between two or more collagen fibrils. Examples include recombinant human decorin core protein (“decorin”).

“Decorin” includes any of the proteins known to the skilled artisan by that name, so long as the decorin functions as a bidentate ligand attached to two or more neighboring collagen molecules in a fibril or in adjacent fibrils. “Decorin” includes the decorin core protein and the proteoglycan form unless the context makes clear that only the protein lacking the glycan is meant. In particular, decorin proteins include those proteins encoded by any of the various alternatively spliced transcripts of the human decorin gene described by REFSEQ number NM_{—}001920.3. In general, the human decorin protein is 359 amino acids in size, and its amino acid sequence is set forth in REFSEQ number NP_{—}001911. Various mutations and their effect on the interaction of decorin with collagen have been described, for example by Nareyeck et al., Eur. J. Biochem., Vo. 271, pages 3389-98 (2004), and those mutants that bind collagen are also within the scope of the term “decorin,” as is the decorin variant known as the 179 allelic variant, De Cosmo et al., Nephron, Vol. 92, pages 72-76 (2002). Decorin for use in the methods of the invention may be purified from various animal tissue sources, or it may be produced recombinantly. Thus, not only human decorin, but decorin from other species, including, but not limited to, primates, cows, pigs, sheep, guinea pigs, mice, and rats, may also be used in the methods of the invention. An example of human decorin that can be used in the methods of the invention is the recombinant human decorin that is available commercially from Catalent Pharma Solutions. Glycosylated or unglycosylated forms of decorin can be used. Fragments of decorin, whether recombinant or proteolytic fragments, may also be employed in certain embodiments so long as those fragments function as a bidentate ligand attached to two or more neighboring collagen molecules in a fibril or in adjacent fibrils.

As used herein, the terms “treatment,” “treating,” and the like, refer to obtaining a desired pharmacologic and/or physiologic effect. A treatment can administer a composition or product to a patient already known to have a condition. A treatment can also administer a composition or product to a patient as part of a prophylactic strategy to inhibit the development of a disease or condition known to be associated with a primary treatment. In the context of a surgical procedure, prophylactic treatment is any treatment administered to a patient scheduled to undergo a surgical procedure for the purpose of improving the outcome of that surgical procedure or otherwise reducing undesirable secondary effects associated with the surgical procedure. An example of a prophylactic treatment is the administration of an immunosuppressive agent to a patient prior to the transplantation of an organ or tissue. “Treatment,” as used herein, covers any treatment of a condition or disease in a mammal, particularly in a human, and includes: (a) inhibiting the condition or disease, such as, arresting its development; and (b) relieving, alleviating or ameliorating the condition or disease, such as, for example, causing regression of the condition or disease.

A “pharmaceutically acceptable carrier” refers to a non-toxic solid, semisolid, or liquid filler, diluent, encapsulating material or formulation auxiliary of any conventional type. A “pharmaceutically acceptable carrier” is non-toxic to recipients at the dosages and concentrations employed and is compatible with other ingredients of the formulation. For example, the carrier for a formulation containing polypeptides preferably does not include oxidizing agents and other compounds that are known to be deleterious to polypeptides. Suitable carriers include, but are not limited to, water, buffer solutions such as Balanced Salt Solution, dextrose, glycerol, saline, cellulosics such as carboxymethylcellulose or hydroxypropylmethylcellulose, polysaccharides such as hyaluronic acid, and combinations thereof. The carrier may contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents may be added as necessary. Other examples of pharmaceutically acceptable carriers are presented throughout the specification, including in the examples.

Pharmaceutically acceptable salts suitable for use herein include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The terms “individual,” “subject,” “host,” and “patient,” used interchangeably herein, refer to a mammal, including, but not limited to, murines, simians, humans, felines, canines, equines, bovines, porcines, ovines, caprines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### Proteins That Crosslink and Organize Collagen Fibrils in ECM

Collagen fibrils can be crosslinked by indirect bonds. In these embodiments of the invention, one or more proteins serve as an intermediary link between or among the collagen fibrils. Decorin is an example of a protein that crosslinks collagen fibrils by indirect bonds. For use in the methods of the invention, decorin is generally dissolved or suspended in a physiologically compatible buffer solution. The concentration of decorin may range from about 10 to about 5000 μg/ml. In some embodiments, the concentration ranges from about 10 to about 500 μg/ml, while in other embodiments it may be from about 100 to about 5000 μg/ml. In still other embodiments the concentration ranges from about 100 to about 1000 μg/ml, about 200 to about 900 μg/ml, about 300 to about 800 μg/ml, about 350 to about 700 μg/ml, about 400 to about 700 μg/ml, or about 400 to about 600 μg/ml. Other proteins that indirectly link collagen fibers by forming a bridge between or among collagen fibrils may be used at the concentrations described for decorin.

The buffer used as a carrier for a protein that forms an indirect crosslink between collagen fibrils is not critical and may be any of a number of pharmaceutically acceptable buffers, such as a neutral pH phosphate buffer. Other suitable buffers include HEPES, TRIZMA® (Sigma-Aldrich, but any other supplier of TRIS buffer should also be acceptable). The buffer will generally have a concentration from about 0.005 to about 0.5M at a pH ranging from about 6.5 to about 8.5, although in some embodiments the pH is from about 6.8 to about 7.6, while in other embodiments the pH is from about 7.0 to about 7.4.

An example of a decorin solution for use in the methods of the invention is one that is sterile and non-pyrogenic, and in which decorin is present at a concentration of about 500 μg/ml and is buffered with 10 mM sodium phosphate plus 15 mM NaCl having a pH of about 7.2. Other proteins that indirectly link collagen fibers by forming a bridge between or among collagen fibrils may be used in this formulation as well.

### Method of Administering Proteins That Crosslink and Organize Extracellular Matrix in Eye Tissues

Various methods can be used to apply a protein that crosslinks and organizes collagen fibrils in a tissue matrix, such as Bruch's membrane. In one embodiment, a solution comprising a protein that crosslinks collagen fibrils is applied by minimally invasive subconjunctival injection, traditional intravitreal injection, transcleral delivery, sub-tenon's injection, supra-choridal delivery, or by other appropriate methods to deliver the solution containing protein to tissue layers at the back of the eye. The injection techniques may involve the use of micro-needles, such as 38 gauge micro-needle or other appropriate gauge micro-needles, or direct injection into Bruch's membrane using micro-needles, such as a 38 or other appropriate gauge micro-needle.

In other embodiments, the protein that crosslinks collagen fibrils may be applied by injection into the scleral tissue. In certain embodiments, the delivery may be by transcleral delivery using a collagen implant that is impregnated with decorin and then placed in the conjunctiva. This delivery technique is distinct from topical administrations because topical administrations are rapidly cleared from the eye, generally via the tear ducts, and so do not provide sufficient contact time with the scleral tissue.

### Patient Selection

The methods of the invention vary in part depending upon the stage of the macular degeneration. Thus, in certain embodiments the methods of preventing development of age-related macular degeneration, diabetic retinopathy, and diabetic macular edema involve administration of a disclosed composition to a patient in which specific signs or symptoms are absent. Signs or symptoms of dry AMD include one or more of loss of pigment in the retina, the presence of drusen, or geographical atrophy of the retina pigment epithelium. Symptoms of wet AMD include loss of central vision, vision distortion, decreased contrast sensitivity, and decreased color vision. Symptoms of diabetic retinopathy include microaneurysms in the retina's blood vessels that could lead to more severe forms wherein new, abnormal, thin-walled and fragile-walled blood vessels grow to supply blood to the retina, but which new blood vessels may leak blood to produce severe vision loss and blindness. Hemorrhages can occur more than once, often during sleep. Fluid can also leak into the center of the macula at any stage of diabetic retinopathy and cause macular edema and blurred vision. Symptoms of diabetic macular edema include a thickening of the retina and/or hard exudates within 1 disc diameter of the center of the retina. a thickening of the retina and/or hard exudates within 1 disc diameter of the center of the retina. Neovascularization originating from the choroids is a sign or symptom of wet AMD, diabetic retinopathy, and diabetic macular edema as is disorganization of Bruch's membrane, including separation of retinal pigment epithelial cells from Bruch's membrane. Accordingly, in one aspect, methods or preventing dry AMD administer a disclosed composition to a patient that lacks a sign or symptom of either dry AMD or wet AMD. In other aspects of the invention, methods of preventing wet AMD, or of preventing the progression of or retarding the development of wet AMD, provide for the administration of a disclosed composition to a patient that has one or more signs or symptoms of dry AMD but no signs or symptoms of wet AMD. In still other embodiments, methods of treating or retarding dry AMD involve administering a disclosed composition to a patient that has one or more signs or symptoms of dry AMD but does not exhibit any signs or symptoms of wet AMD, for example the patient does not exhibit neovascularization in the choroids. Similarly, in other embodiments, methods of preventing diabetic retinopathy and diabetic macular edema, or of preventing the progression of diabetic retinopathy and diabetic macular edema provide for the administration of a disclosed composition to a patient that has one or more signs or symptoms of these blinding conditions.

The methods have been described generally with respect to their method steps and the compositions used. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a”, “an” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Unless defined otherwise, all technical and scientific terms used herein have the same meaning as commonly understood by one of ordinary skill in the art to which this invention belongs. All publications mentioned herein, including patents, patent applications, and publications are incorporated herein by reference in their entireties to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention described below is given by way of example only and is not to be interpreted in any way as limiting the invention.

Reference will now be made in detail to the present embodiments of the invention.

### Examples

### Example 1

### Measurement of Intravitreal Injection of Decorin in the Rabbit Model

The rabbit vitreal Replacement Bioassay was conducted at Insight Biomed (Isanti, Minn.). Six rabbit eyes received intravitreal injection of 0.5 mL of decorin (4.74 mg/mL in 10 mM NaPO₄+150 mM NaCl pH 7.0 following removal of an equal volume of vitreous humor. Contralateral eyes served as a non-operated control. Animals were monitored post-operatively until recovery from anesthesia. At 48 hours the animals were anesthetized by intramuscular injection of Xlyazine (10 mg/Kg body weight), Ketamine (50 mg/kg body weight), and Acepromazine (0.5 mg/kg body weight). Eyes were dilated using topical 2.5% phenylephrine HCl and 1% tropicamide. At 48 hours post injection, both control and treated eyes were graded for ocular inflammation as per the Rabbit Vitreal Grading Scale prior to removal of a vitreal sample. Proparacaine 0.5% was administered topically prior to removal of the 0.5 ml vitreal test sample. Cell counts and ocular inflammatory responses were determined to evaluate the inflammatory response to the test sample.

Test material is considered non-inflammatory if the vitreal cell count is ≦100 cells/mm³ and/or the overall mean clinical response is less than or equal to 1. Non-operated eyes must also be scored non-inflammatory as defined by the parameters of the bioassay.

All six eyes after intravitreal injection with decorin (4.74 mg/mL) exhibited an overall mean vitreal cell count of 2.92 cells/mm³ and an overall mean clinical response of 0.28. Based on these parameters, the test solution, decorin solution, is considered non-inflammatory.

### Example 2

### Angiogenesis Inhibition Test-CAM ASSAY

Recent studies conducted for Euclid Systems have clearly demonstrated the anti-angiogenic activity of human recombinant decorin core protein. Anti-angiogenic activity was demonstrated in the standard chorioallantoic membrane (CAM) in fertilized eggs. Study conducted by MB Research Laboratories, Spinnerstown, Pa. (Project No.10-18858.09). Results are shown below demonstrating a dose related inhibition of vascularization.

TABLE 1
Inhibition of vascularization from CAM Assay

| | | | | |
|---|---|---|---|---|
| Concentration | 0 | 1.22 mg/mL | 2.42 mg/mL | 4.87 mg/mL |
| % Inhibition | 0 | 0.188 | 0.20 | 0.5 |

### Example 3

### Anti-angiogenesis Cell Tube Assay

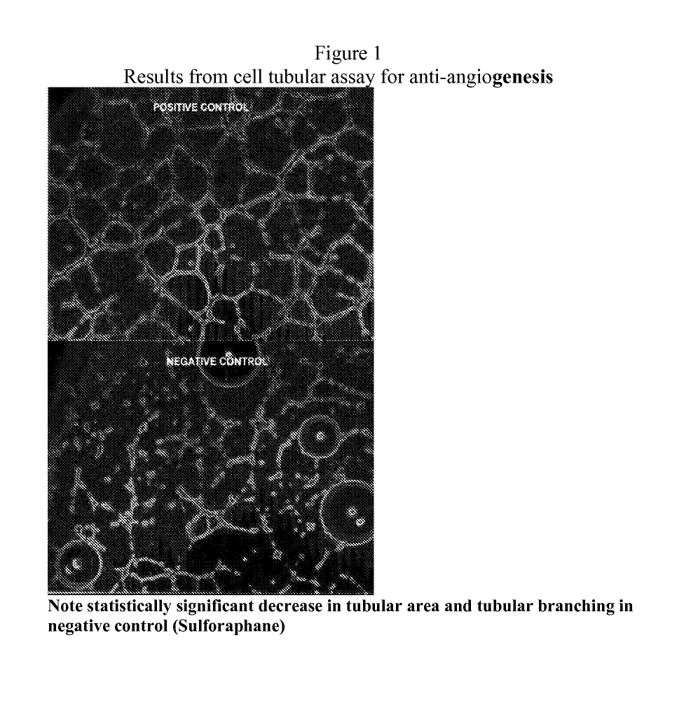

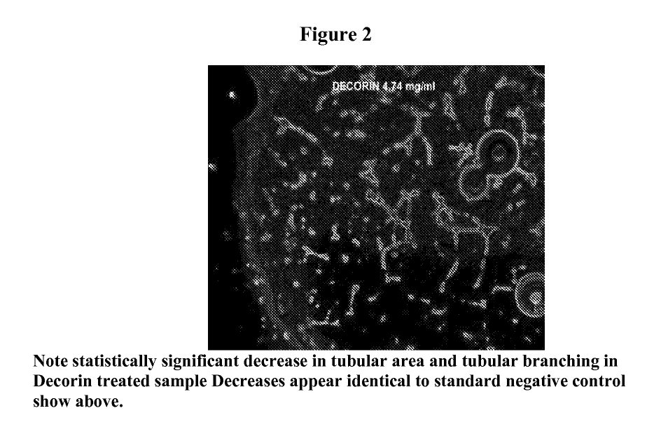

Additional studies evaluated the anti-angiogenic properties of decorin in the Matrigel Tube Formation Assay. This assay is one of the most specific tests for angiogenesis and measures the ability of endothelial cells to form three-dimensional structures (tube formation). Inhibition of tube formation is directly related to anti-angiogenesis activity. Decorin was found to be a significant inhibitor of tube formation, equal to or even more inhibitory than the negative control. It is believed that inhibition may be associated with stabilization of the endothelial cell layer as well as inhibiting growth factor stimulated angiogenesis. (see FIG. 1)

### Example 4

### Stabilization of Bruch's Membrane (Prophetic)

The D-Galactose-Treated Mouse Model is used to determine the effect of decorin on stabilizing and reorganizing the extracellular matrix of Bruch's membrane. Female C57BL/6 mice (4 months old) are purchased from Charles River (Wilmington, Mass.). The animals are housed in plastic cages, kept in a 12-hour light-dark cycle and given a 4 week adaptation period. Group 1, consisting of 5-month old animals are sacrificed at the start of the study. Group 2 animals are given daily s.c. injections of D-galacotose, 50 mg/kg for 4 weeks. Group 3 animals are given daily s.c. injections of D-galacotose, 50 mg/kg for 8 weeks. Group 4 animals are treated with Phosphate Buffered Saline (PBS) for 4 weeks and Group 5 animals are treated with Phosphate Buffered Saline (PBS) for 8 weeks. Group 6 animals are given daily s.c. injections of D-galacotose, 50 mg/kg for 3 weeks and then treated with daily intravitreal injections (0.1 ml) of decorin (4.5 mg/ml) for 1 week. Group 7 are given daily s.c. injections of D-galacotose, 50 mg/kg for 7 weeks and then treated with daily intravitreal injections (0.1 ml ) of decorin (4.5 mg/ml) for 1 week.

After sacrifice at 4 weeks or 8 weeks, eyes are enucleated and one eye placed in 2% paraformaldehyde and 2.5% Glutaraldehyde in 0.08 M cacodylate buffer, pH 7.3. The lens and RPE/choroid of the contralateral eye of each animal are dissected and cryopreserved. The central 2×2 mm tissue temporal to the optic nerve is used for electron microscopy. Fixed tissue is postfixed in 1% osmium tetroxide in 0.1 M cacodylate buffer, dehydrated, embedded in Poly/Bed and sectioned to prepare 1.0 p specimens. Specimens are stained with toluidine blue in 2% sodium borate. Ultrathin sections are cut, stained with uranyl acetate and lead citrate and examined by electron microscopy.

Electron microscopy evaluation shows an increase in Bruch's membrane thickness and membrane disorganization after D-galactose treatment at 4 and 8 weeks, with greater thickening observed at the 8 week time point. Conversely, electron microscopy evaluation shows normal Bruch's membrane thickness and normal Bruch's membrane organization in decorin treated animals (same as control specimens) at 4 weeks and nearly normal Bruch's membrane thickness and normal Bruch's membrane organization in decorin treated animals (same as control specimens) at 8 weeks. This study is expected to demonstrate the effectiveness of decorin in retarding or reversing the development of macular degeneration in this animal model. Similar effects are expected in retarding the development of human, dry AMD or reversing critical events associated with human, dry AMD.

### Example 5

### Preservation of Cell Structure

Recent studies conducted for Euclid Systems have demonstrated the ability of decorin to preserve corneal endothelial and epithelial cell layers, to increase mean cell density, and improve intracellular junctures (Insight BioMed Reports 09PHCE-Eu01/001, 002, 003). Application of exogenously applied decorin to the RPE layer on Bruch's membrane will stabilize this critical cell layer and prevent development or further development of choroidal neovascularization. Such cell stabilization will also reduce production of angiogenic growth factors (such as VEGF) associated with RPE cell disruptions that precede choroidal neovascularization.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### LITERATURE REFERENCES

Ida, H, Ishibashi, K, Reiser, K, Hjelmeland, L M, and Handa, J T. Ultrastructural aging of the RPE-Bruch's Membrane-Choriocapillaris complex in D-galactose-treated mouse. 2004. IOVS, 45:2348-2354.
Iwasaki, S, Hosaka, Y, Iwasaki, T, Yamamota, K, Nagayasu, A, Ueda, H, Kokai, Y, and Takehana, T. The modulation of collagen fibril assembly and its structure by decorin: An electron microscopic study, Arch. Histol. Cystol. 2008. 71:37-44
Iozzo, R V. The biology of the small leucine-rich proteoglycans. 1999. J. Biol. Chem. 274:18843-18846

Zarbin, M A. Current concepts in the pathogenesis of age-related macular degeneration. 2004. Arch Ophthalmol. 122: 598-614

Orgel, PRO, Eid, A, Antipova, O, Bella, J, and Scott, J E. Decorin core protein (Decoron) shape complements collagen fibril surface structure and mediates its binding. 2009. PLoS One, 4:1-10

Cook, H L, Patel, P J, and Tufail, A. Age-related macular degeneration: diagnosis and management. 2008. Brit. Medical Bulletin, 85: 127-149

Sherman, J and Aswani, M. Age related macula degeneration: Novel diagnostic and treatment options. SUNY College of optometry

Zarbin, M and Szirth, B. Current treatment of age-related macular degeneration. 2007. Optometry and Vision Science. 84: E559-E572

Chopdar, A, Chakravarthy, U, and Verma, D. Age related macular degeneration.

### 2003. BMJ. 326: 485-488

Tasheva, E S, Ke, A, and Conrad, G W. Analysis of the expression of chondroadherin in mouse ocular and non-ocular tissues. 2004. Molecular Vision. 10: 544-554
Inatani, M, Tanihara, H, Honjo, M, Hangai, M, Kresse, H., and Honda, Y. Expression of proteoglycan decorin in neural retina. 1999. Investigative Ophthalmology & Visual Sci., 40: 1783-1791.
Grant, D S, Yenisey, C, Rose, R W, Tootell, M, Santra, M, and lozzo, R V. Decorin suppresses tumor cell-mediated angiogenesis. 2002. Oncogene 21:4765-4777
Sconherr, E, Sunderkotter, C, lozzo, R V, and Shaefer, L. Decorin, a novel player in the insulin-like growth factor system. 2005. J. Biol. Chem. 280: 15767-15772
Sulochana, K Fan, H, Jois, S, Subramanian, V, Sun, F, Kin, M, and Ge, R. Peptides derived from human decorin leucine-rich repeat 5 inhibit angiogenesis. 2005. J. Biol. Chem. 280:27936-27948
Hirabayashi, Y, Fujimori, O, and Shimizu, S. Bruch's membrane of the brachymorphic mouse. 2003, Medical Electron Microscopy, 36: 136-146
Scott, J E, The structure of interfibrillar proteoglycan bridges (shape modules) in extracellular matrix of fibrous connective tissues and their stability in various chemical environments, 1998. J. Anatomy. 192: 391-405
Scott, J E, Proteodermatan and proteokeratan sulfate (decorin, lumican/fibromodulin) proteins are horseshoe shaped. Implications for their interactions with collagen.1996 Biochemistry, 35: 8795, 1996
Scott, J E, Dyne, K N, Thomlinson, K L, Richie, M, Cetta, G, and Valli, M. Human cells unable to express decoron produced disorganized extracellular matrix lacking “shape modules” (interfibrillar proteoglycan bridges. 1998 Exp. Cell Res., 243: 59-66
Rada, J A, Cornuet, PK and Hassell, J R. Regulation of corneal collagen fibrillogenesis in vitro by corneal proteoglycan (lumican and decorin) core proteins. 1993. Exp Eye Res., 56: 635-648
Kuc, I M and Scott, P G, Increased diameters of collagen fibrils precipitated in vitro in the presence of decorin from various connective tissues. 1997. Connective Tissue Res., 36: 287-296
Giri, S N, Hyde, D M, Braun, R K, Gaarde, W, Harper J R, and Pierschbacher, M D. 1997. Antifibrotic effect of decorin in a bleomycin hamster model of lung fibrosis. Biochem. Pharmacol., 54: 1205-1216.
Westergen-Thorsson, G, Hernnas, J., Sarnstrand, B, Oldberg, A., Heinegard, A. and Malmstrom, A. Altered expression of small proteoglycans, collagen, and transforming growth factor-beta 1 in developing bleomycin-induced pulmonary fibrosis in rats. 1993 .J. Clin. Invest., 92: , 632-637.
Gu, J. and Wada, Y., Effect of exogenous decorin on cell morphology and attachment of decorin-deficient fibroblasts. 1996. J. Biochem (Tokyo), 119: 743-748
Nash, M A, Loercher, A E and Freedman, R S. In vitro growth inhibition of ovarian cancer cells by decorin: synergism of action between decorin and carboplatin. 1999. Cancer Res., 59: , 6192-6196
Sulochana, K Fan, H, Jois, S, Subramanian, V, Sun, F, Kin, M, and Ge, R. Peptides derived from human decorin leucine-rich repeat 5 inhibit angiogenesis. 2005. J. Biol. Chem. 280:27936-27948
   Kwak, N, Okamoto, N, Wood, J M, and Campochiaro, VEGF is major stimulator in model of choroidal neovascularization. 2000. Invest. Ophthal. Vis. Sci. 41: 3158-3164.
Bonilha, V L. Age and disease-related structure changes in the retinal pigment epithelium. 2008. Clinical Ophthalmology 2:413-424
Sun, K, Cai, H, Tezel, T H, Paik, D, Gaillard, E R, and Del Priore, L V. Bruch's membrane aging decreases phagocytosis of outer segments by retinal pigment epithelium. 2007. Molecular Vision. 13:2310-2319.
Fell, A J, Engelmann, K, Richard, G, Fassbender, C, Wahls, W, and Klingel, R. Rheophoresie. A systematic approach to therapy of age-related macular degeneration. 2002. Ophthalmologe. 99:780-784
Del Priore, L. Role of Bruch's membrane aging in RPE behavior: What have we learned from transplantation studies. 2005. Invest. Ophthalmol. Vis. Science. 46-Abstract 1212.
Tezel, T H, Sonmez, K, Del Priore, L V, Kaplan, H J. Tissue reconstruction in AMD: Breaking the collagen crosslinks increases RPE reattachment rate onto aged inner Bruch's membrane. 2005. ARVO May 1-5

What is claimed is:

1. A method of treating the dry form of age-related macular degeneration (AMD) or preventing the development of the wet form of AMD in a patient in need thereof, the method comprising administering a pharmaceutical composition comprising decorin and a pharmaceutically acceptable carrier by an injection technique to the back of the eye of a patient having the dry form of AMD, to thereby treat the dry AMD or prevent the development of wet AMD.

2. A method of treating diabetic retinopathy or preventing the development of diabetic macular edema in a patient in need thereof, the method comprising administering a pharmaceutical composition comprising decorin and a pharmaceutically acceptable carrier by an injection technique to the back of the eye of a patient having diabetes, to thereby treat the diabetic retinopathy or prevent the development of diabetic macular degeneration.

3. A pharmaceutical composition comprising decorin and a pharmaceutically acceptable carrier for use in treating the dry form of age-related macular degeneration (AMD) or preventing the development of the wet form of AMD in a patient in need thereof.

4. A pharmaceutical composition comprising decorin and a pharmaceutically acceptable carrier for use in treating diabetic retinopathy or preventing the development of diabetic macular edema in a patient in need thereof.

5. Use of a composition comprising decorin and a pharmaceutically acceptable carrier for treating the dry form of age-related macular degeneration (AMD) or preventing the development of the wet form of AMD in a patient in need thereof.

6. Use of a composition comprising decorin and a pharmaceutically acceptable carrier for treating diabetic retinopathy or preventing the development of diabetic macular edema in a patient in need thereof.

7. The method of claim 1 or 2, the pharmaceutical composition of claim 3 or 4, or the use of claim 5 or 6, wherein the decorin is a recombinant human decorin core protein.

8. The method of claim 1 or 2, wherein the decorin is applied by minimally invasive subconjunctival injection or by traditional intravitreal injection.

9. The method of claim 8, wherein the decorin is applied directly to Bruch's membrane.

10. The method of claim 1 or 2, wherein the decorin is applied transclerally using a collagen impregnated delivery system.

11. A method of stabilizing collagen fibrils in an extracellular matrix of retinal tissue of a patient experiencing the dry form of age related macular degeneration, comprising administering a composition comprising a proteoglycan molecule, or the core protein of a proteoglycan molecule, that crosslinks collagen fibrils and a pharmaceutically acceptable carrier to the eye of the patient.

12. A method of organizing collagen fibrils in an extracellular matrix of retinal tissue of a patient experiencing the dry form of age related macular degeneration, comprising administering a composition comprising a proteoglycan molecule, or the core protein of a proteoglycan molecule, that crosslinks collagen fibrils and a pharmaceutically acceptable carrier to the eye of the patient.

13. A method of stabilizing and organizing collagen fibrils in the extracellular matrix of Bruch's membrane of patients experiencing the dry form of age related macular degeneration, comprising administering a composition comprising a proteoglycan molecule, or the core protein of a proteoglycan molecule, that crosslinks collagen fibrils and a pharmaceutically acceptable carrier to the eye of the patient.

14. A method of stabilizing and organizing collagen fibrils in the extracellular matrix of Bruch's membrane of patients experiencing mild dry age related macular degeneration to retard or limit the development of the wet form of age related macular degeneration comprising administering a composition comprising a proteoglycan molecule, or the core protein of a proteoglycan molecule, that crosslinks and organizes collagen fibrils and a pharmaceutically acceptable carrier to the eye of the patient.

15. The method of claim 14, wherein composition prevents the development of wet macular degeneration

16. A method of treating dry age related macular degeneration and preventing or minimizing the development of wet age related macular degeneration comprising administering a composition comprising a proteoglycan molecule, or the core protein of a proteoglycan molecule, that crosslinks and organizes collagen fibrils and a pharmaceutically acceptable carrier to the eye of the patient.

17. The method of claim 16, wherein the proteoglycan or the core protein of the proteoglycan also inhibits angiogenesis to retard development of wet age-related macular degeneration.

18. The method of claim 16, wherein proteoglycan or the core protein of the proteoglycan prevents the development of macular degeneration.

19. A method of preventing macular degeneration in a patient comprising administering a composition comprising a proteoglycan molecule, or the core protein of a proteoglycan molecule, that crosslinks and organizes collagen fibrils and a pharmaceutically acceptable carrier to the eye of the patient.

20. The method of claim 19, wherein the proteoglycan or the core protein of the proteoglycan also inhibits angiogenesis to retard development of wet age related macular degeneration.

21. A method of treating diabetic retinopathy and preventing or minimizing the development of diabetic retinopathy comprising administering a composition comprising a proteoglycan molecule, or the core protein of a proteoglycan molecule, that crosslinks and organizes collagen fibrils and stabilizes retinal pigment epithelial cell layers attached to Bruch's membrane and a pharmaceutically acceptable carrier to the eye of the patient.

22. A method of treating diabetic retinopathy and preventing or minimizing the development of diabetic macular edema comprising administering a composition comprising a proteoglycan molecule, or the core protein of a proteoglycan molecule, that crosslinks and organizes collagen fibrils and stabilizes retinal pigment epithelial cell layers attached to Bruch's membrane and a pharmaceutically acceptable carrier to the eye of the patient.

23. The method of any one of claims 11-22, wherein the proteoglycan or the core protein of the proteoglycan is chosen from decorin, biglycan, epiphycan, keratocan, mimican, fibromodulin, lumican, or any combination thereof.

24. The method of any one of claims 11-22, wherein the proteoglycan or the core protein of the proteoglycan is a recombinant human decorin.

25. The method of any one of claims 11-22, wherein the composition is applied by minimally invasive subconjunctival injection or by traditional intravitreal injection.

26. The method of any one of claims 11-22, wherein the composition is applied by minimally invasive subconjunctival injection or by traditional intravitreal injection into Bruch's membrane.
